(19) United States
(12) Patent Application Publication
(10) Pub. No.: US 2011/0129520 A1
(43) Pub. Date: Jun. 2, 2011

(54) Anti-Adhesion Barrier Wound Dressing Comprising Processed Amniotic Tissue and Method of Use

(76) Inventors: Simon Bogdansky, Littleton, CO (US); Chad Ronholdt, Aurora, CO (US); Adrian Samaniego, Louisville, CO (US); Kenneth Blood, Littleton, CO (US); Terrell Suddarth, Winchester, TN (US)

(21) Appl. No.: 12/428,836

(22) Filed: Apr. 23, 2009

Related U.S. Application Data
(60) Provisional application No. 61/047,842, filed on Apr. 25, 2008.

(51) Int. Cl. A61L 15/16; A61K 35/48

(52) U.S. Cl. 424/446; 424/582

(57) ABSTRACT

An anti-adhesion wound barrier fabricated from amnion obtained from human birth tissue and treated with a glutaraldehyde solution is provided. The amnion is treated in 1% glutaraldehyde solution for up to 15 minutes to fix the amnion. Methods of processing the birth tissue to prepare the amnion for use as a wound barrier are also provided. Use of the amnion anti-adhesion wound barrier for dressing wounds is also described.

This application claims priority under 35 U.S.C. §120 to U.S. Provisional Application No. 61/047,842 filed on Apr. 25, 2008, the entire contents of which are hereby incorporated by reference.

### FIELD OF THE INVENTION

The present invention is directed, in one aspect, to anti-adhesion wound dressings fabricated from amnion obtained from human birth tissue and treated with a glutaraldehyde solution. In another aspect, the present invention is directed to methods of processing the birth tissue to prepare the amnion for use as a wound dressing. In yet another aspect, the present invention is directed to methods of using the processed amnion for dressing a wound.

### BACKGROUND OF THE INVENTION

Postoperative fibrosis is a natural consequence of all surgical wound healing. The source of fibrotic tissue after spinal surgery was originally thought to be the disrupted intervertebral disc but a later study revealed that fibroblasts arose from the disrupted epaxial muscles in the surgical wound. Postoperative peridural adhesion results in tethering, traction, and compression of the thecal sac and nerve roots, which cause a recurrence of hyperesthesia that typically manifests a few months after laminectomy surgery. Although controversy exists about the role of peridural fibrosis in failed-back syndrome, it is accepted by many to be a problematic clinical entity with no efficacious treatment options. Repeated surgery for removal of scar tissue is associated with poor outcome and increased risk of injury because of the difficulty of identifying neural structures that are surrounded by scar tissue. Therefore, experimental and clinical studies have primarily focused on preventing the adhesion of scar tissue to the dura matter and nerve roots.

More than 300,000 cervical and lumbar laminectomies are performed each year and approximately 10% require revision. Spinal adhesions have been implicated as a major contributing factor in failure of spine surgery. Fibrotic scar tissue can cause compression and tethering of nerve roots, which can be associated with recurrent pain and physical impairment. During the revision procedure, the scar tissue from the original surgery must be painstakingly dissected away from the dura. This dissection process can add hours to the revision surgery thereby creating undue stress on the surgeon as well as the patient who must remain under anesthesia for an extended timeframe. Tearing of the dura can occur during this dissection, resulting in spinal fluid leak. Implantation of a anti-adhesion barrier during the original surgery would allow for an easier exposure of the dura should a revision surgery become necessary.

The ideal agent for preventing peridural adhesion and fibrosis would have the following properties: 1) prevention of scar tissue adhesion to the dural tissues; 2) prevention of the development of leptomeningeal arachnoiditis; 3) no potential to impair dural healing following tearing and CSF leakage; and 4) no capability to induce excessive inflammation around neural tissues. Previously studied materials or procedures include autografts (free and pedicled fat grafts, ligamentum flavum, and lamina replacement); manufactured biomaterials that provide a mechanical barrier (for example, expanded polytetrafluoroethylene membrane, Gelfoam, Sialastic membrane, Surgicel, Avitene, polymethyl methacrylate, TachoComb, synthetic carbohydrate polymers, and Goretex); topical administration of biochemicals to reduce fibroblast function and infiltration (for example, urokinase, tissue plasminogen activator, mitomycin-C, hyaluronic acid, and glucocorticoids); and intraoperative application of CO2 laser therapy or localized administration of external-beam radiation therapy perioperatively. The effectiveness and safety of each of these agents and procedures have not met with widespread acceptance.

Use of free fat autografts as an interposition membrane is probably the most common practice in spinal surgery performed in humans, but some reports have shown little benefit or even detrimental results caused by herniation of the fat graft and subsequent neural impingement. Previously, investigators have suggested that the most statistically and consistently effective antiadhesion barrier used in spinal surgery was ADCON-L. ADCON-L is a gel that is generally comprised of marginally water soluble artificial sugars. Multiple reports have been published that implicates ADCON-L in impaired dural healing and persistent CSF leakage in humans. These complications have been linked to inadvertent or unrecognized intraoperative dural tearing despite experimental work in rats that indicated that ADCON-L would not impair dural healing.

Human amnionic membrane has been used for many years in various surgical procedures, including skin transplantation and ocular surface disorders. Amionic tissue is obtained from human birth tissue, which is defined as the amniotic sac (comprised of two tissue layers; amnion and chorion), placenta, the umbilical cord and the cells and fluids contained within each. The material provides good wound protection, can reduce pain, reduce wound dehydration, and provide anti-inflammatory and anti-microbial effects. The amnion is the innermost layer of the placental membranes. It is a thin semi-transparent membrane normally 20 μm to 500 μm in thickness. The amnion comprises a single layer of ectodermally derived columnar epithelial cells adhered to a membrane comprised of collagen I, collagen III, collagen IV, laminin and fibronectin which in turn is attached to an underlying layer of connective tissue. The connective tissue is comprised of an acellular compact layer of reticular fibers, a fibroblast layer and a spongy layer consisting of a network of fine fibrils surrounded by mucus.

Various processes have been used to treat human amniotic tissue to enhance its physical properties and to provide a material that can be stored for easy use. For example, Baur, U.S. Pat. No. 4,361,552 describes treatment of amnion using a solution containing a fixing agent such as gluteraldehyde. At column 3, lines 3-19, Baur describes treating amnion for periods of time ranging from a few hours for solutions containing up to 25% gluteraldehyde to several weeks for weaker solutions. Treatment of amnion for use in reconstruction of ocular surfaces is described in Spoerl, “Cross-linking of Human Amniotic Membrane by Gluteraldehyde”, Opthalmic Research, 2004, 36:71-77. Spoerl describes treatment of amnion in a 0.1% gluteraldehyde/20% dextran solution for a period of 30 minutes. Spoerl states that a 0.1% gluteraldehyde concentration was used due to the cytotoxicity of gluteraldehyde. The amnion in Spoerl was washed for 30 minutes prior to use in a dextran/saline solution.

While these processes have had varying degrees of success, there remains a need for an improved process for treating human amniotic material to produce a material having improved properties for use in treating spinal surgical wounds as wells as wounds caused by other traumatic or degenerative events.

### BRIEF DESCRIPTION OF THE FIGURES

FIG. 1 shows a flow chart for one embodiment of the process for treating human amniotic tissue.

### SUMMARY OF THE INVENTION

The present invention is generally directed to an amnion anti-adhesion wound dressing, referred to herein as an Amnion Anti-adhesion Bather (“AAB”), and processes for producing the AAB material. The AAB has unique properties that surprisingly prevent fibrous scar formation when implanted post-operatively into a human for a variety of surgical procedures. The AAB is a thin, translucent, acellular membrane that can be cut into a variety of sizes for virtually any post-operative surgical procedure where anti-scar formation is desired (e.g. such as but not limited to spine surgery, knee surgery, child birth, shoulder surgery, trauma related cases, cardiovascular procedures, brain/neurological procedures, burn and wound care etc.). In a preferred use of the material, it is used as an anti-adhesion dressing in spinal surgeries. The AAB is fabricated from human birth tissue, which is defined as the amniotic sac (comprised of two tissue layers; amnion and chorion), placenta, the umbilical cord and the cells and fluids contained within each.

The AAB performs the very critical function in-situ of providing a immunoprivileged environment (i.e. relatively high resistance against immune responses) in the human development process that make it uniquely suited for its anti-scar adhesion duties. The distinct surface and molecular architecture characteristics of the tissue are what make this tissue immunoprivileged and when transplanted there are no immunologic markers (e.g., antibodies, antigens) present to induce the immunologic cascade which would result in a foreign body reaction and/or rejection of the tissue. The AAB is a highly organized architecture comprised of primarily collagen types I, III, V and VII and glycosaminoglycans (hereafter referred to as GAG's). Depending upon whether or not the chorion was completely removed there may also be small amounts of type IV collagen and proteoglycans that comprise the thicker chorion tissue. It is this highly complex architecture that gives the AAB its anti-scar formation capabilities. The AAB also contains amino acids and molecules/proteins, as well as the innate avascular nature of the tissue, that contribute to another feature, the anti-inflammatory capabilities of the material. The thicker chorion tissue contains all of the vascular vessels and capillaries, nerves and majority of the cells, although a single layer of specialized epithelial cells line the inner most surface of the amnion tissue (i.e. closest to the baby).

The AAB material is produced by processing human amniotic tissue. In one embodiment of the invention, the human amniotic tissue is processed by debriding the tissue to remove the amnion from the chorion and other amniotic tissue and fluids. The amnion is rinsed in a sterile saline solution, such as 0.9% NaCl solution. Preferably, multiple saline rinse steps are performed. After rinsing with sterile saline, the amnion is soaked in a glutaraldehyde solution. Preferably, the amnion is soaked in a 1% glutaraldehyde solution for a period of up to 15 minutes. Following the glutaraldehyde soak, the amnion is again rinsed with sterile saline solution. The treated amnion is covered with a material, such as for example gauze, and the treated amnion is cut to the desired size for a surgical patch. As described in detail below, amnion can be difficult to handle and manipulate to apply the dressing to the surgical site. Accordingly, the packaging of the amnion should facilitate handling the amnion and maintaining the orientation of the amnion patch. Each surgical patch is packaged with saline solution to maintain the moisture of the patch. The packaged amnion is sterilized using irradiation. In one embodiment, the amnion is sterilized using E-beam sterilization in the range of 10-35 kGy, and preferably between 25-35 kGy.

Throughout the procedure described above, the orientation of the amnion is identified to ensure that in use the correct side of the amnion patch is placed on the wound. Either the fetal (epithelial) side or the maternal (stromal) side of the amnion patch may be used depending upon the specific use or procedure that is being performed. In one embodiment, the maternal side of the amnion patch is applied to the spinal dura following spinal surgery.

One advantage of the wound dressings and processes of the present invention is that an anti-adhesion barrier is provided which can be used to prevent adhesions following surgery, and in particular following back surgery. Other advantages of the wound dressings and processes of the present invention will be apparent to those skilled in the art based upon the detailed description of preferred embodiments set forth below.

### DETAILED DESCRIPTION OF PREFERRED EMBODIMENTS

The present invention generally relates to an anti-adhesion wound dressing that is prepared from human birth tissue. In particular, the invention relates to the use of amnion obtained from human birth tissue to form an Amnion Anti-adhesion Barrier (“AAB”) for use as a wound dressing. The invention further relates to methods for aseptically processing amniotic tissue to produce an amnion material that may be used to prepare an AAB wound dressing. The processed amnion provides an anti-adhesion barrier to prevent formation of post-operative surgical adhesions. The AAB wound dressing may be used, for example, for surgery involving the spine, knee, shoulder or child birth, trauma related wounds or injuries, cardiovascular procedures, angiogenesis stimulation, brain/neurological procedures, burn and wound care, ophthalmic procedures or in any other procedure where an anti-adhesion barrier is desirable. In a particularly preferred embodiment, the amnion anti-adhesion barrier is used in spinal surgery.

The preferred embodiment of the AAB is a terminally sterilized amnion anti-adhesion barrier that is stable at room temperature 22° C.) which has been processed, packaged and irradiated at 25-35 kGy using Electron Beam (E-Beam) irradiation for sterilization. The process is such that the integrity and structure of the tissue is not altered and the specialized proteins and amino acids are not denatured or destroyed and if possible concentrated to improve clinical performance. The sterilization step provides the surgeons and patients with a high level of confidence that they are receiving a sterile and safe tissue product which does not adversely affect the molecular or gross structure of the tissue in a manner that would impede its performance.

In the embodiments of the process described below, the following general conditions are preferably followed. Tissues from a specific donor should not be processed with tissues from another donor. Processing should not change the physical properties of the tissue so as to make them unacceptable for clinical use. Instruments, solutions, and supplies coming into contact with tissue during the processing of the amnion should be sterile. All surfaces coming in contact with tissue intended for transplant should be either sterile or draped using aseptic technique. Amnion intended for transplant should be processed as soon as practicable, and in any event within 7-10 days from the date of procurement. Processed amnion should be shipped and sterilized by irradiation according to protocol as soon as practicable, and typically within no more than 3 days of processing. The processing of amnion is performed in a controlled environment (i.e. certified hoods or clean rooms). All instruments, solutions and supplies that come in contact with tissue during processing and packaging should be handled aseptically. The tissue or solutions should be maintained at 1° to 10° C. for amnion prior to and after being processed until time of sterilization. Amnion should be kept moist during processing using a sterile, isotonic solution/medium. It is expected that sterilized amnion may be stored for up to one year from the date of processing, and that the amnion may be stored under proper conditions for as much as five years following processing.

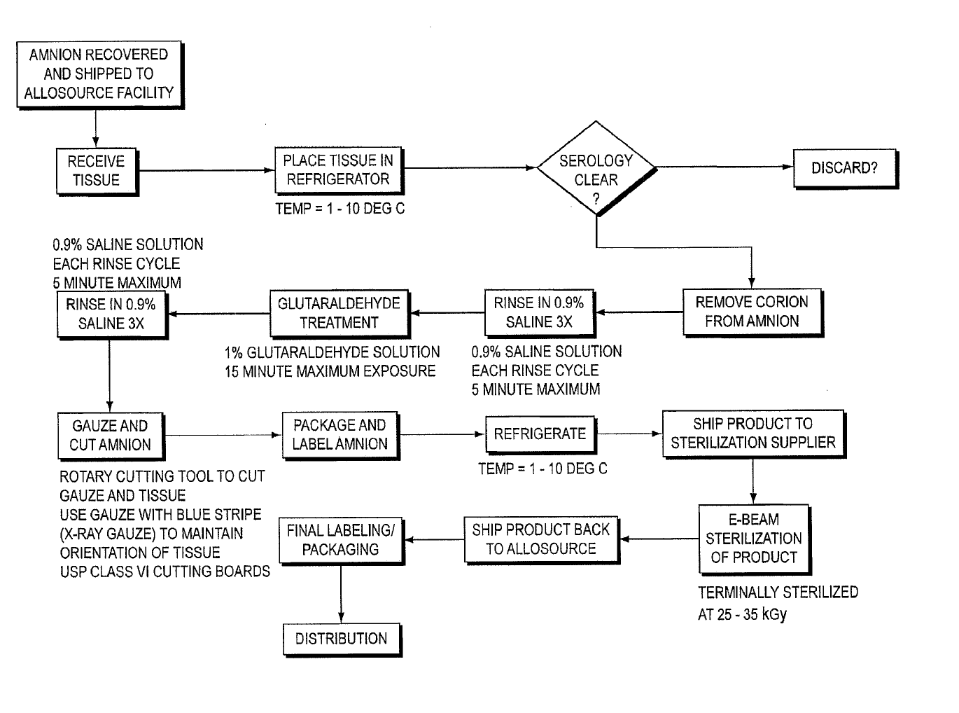

A flow chart showing generally the steps of an embodiment of the process is shown in FIG. 1. Human birth tissue is recovered from a full term cesarean delivery of a newborn. The amnion, chorion, umbilical cord and placenta are recovered after the newborn is removed. The placenta and umbilical cord are removed and discarded. The amnion/chorion is rinsed with sterile saline solution and placed in a container filled with sterile saline solution for shipment. The tissue is shipped on wet ice and stored at a temperature of between 1° C. and 10° C. while serology testing is performed. Upon receipt of acceptable test results, the tissue is processed further.

To prepare the amnion for treatment, the tissue is laid flat with the chorion side up. The chorion/Wharton's jelly is removed by applying finger pressure and sliding the chorion/Wharton's jelly off the amnion using as little pressure as possible to avoid tearing the amnion. The chorion/Wharton's jelly and any excess tissue is discarded.

The orientation of the amnion should be maintained during treatment and the maternal (stromal) and fetal (epithelial) sides of the amnion should be identified. Depending upon the use or treatment being performed, it may be desirable to use a particular side of the amnion for application. Maintaining and identifying the orientation of the amnion allows the physician to identify the proper side to be used for a particular treatment. For example, as described in the sheep model study described in the examples below, in the case of applying amnion to the dura following spinal surgery, it is preferable to place the maternal side of the amnion upon the dura to prevent or minimize scar fibrosis.

In one embodiment, different colored clips are attached to the amnion to identify the maternal and fetal sides of the tissue. Any suitable means of maintaining and identifying the orientation of the tissue may be used. For example, one side of the amnion may be marked with a stamp or ink, or an adherent paper having identifying printing may be used.

The amnion is first rinsed using sterile saline solution. The amnion may be rinsed in bowls or trays of sufficient size to allow the amnion to be spread out to improve the rinse. In a preferred embodiment, the sterile saline solution is a 0.9% NaCl solution. Sufficient saline solution should be used to ensure that the amnion is completely immersed. The contents of the bowl or tray are agitated by gently stirring or swirling in a circular motion to liberate excess blood and bodily fluids from the amnion. The saline is then decanted into a discard basin. Multiple saline rinse cycles may be performed. In one embodiment, the amnion is rinsed for three separate rinse cycles, with each rinse cycle lasting for a maximum of 5 minutes.

Following the saline rinse, the amnion is treated in a 1% glutaraldehyde solution. The glutaraldehyde treatment is preferably performed in a bowl or tray of sufficient size to allow the tissue to spread out to maximize exposure of the tissue to the glutaraldehyde solution. Sufficient gluteraldehyde solution should be used to immerse the amnion in the solution. Typically, a minimum of about 400 ml of gluteraldehyde solution is used. The tissue is soaked in the glutaraldehyde solution for a maximum of 15 minutes with gentle stirring or swirling at a temperature of 22° C. (+1-5° C.).

After the glutaraldehyde treatment, the amnion is again rinsed with sterile saline solution in the same manner as described above. Multiple saline rinses may be performed. In one embodiment, the gluteraldehyde treated amnion is rinsed in a sterile saline solution, such as a 0.9% NaCl solution, for three separate rinse cycles, with each rinse cycle lasting for a maximum of 5 minutes.

Following the second series of saline rinses, the amnion is spread out and covered. The covering used will typically be used in the packaging of the amnion. Because amnion tissue can be difficult to handle and manipulate when applying to a surgical site, the packaging of the amnion and the covering used should facilitate the handling of the amnion and maintaining and identifying the orientation of the fetal and maternal side of the amnion for the user. The packaging may also promote storage of the amnion.

In one embodiment, the amnion is covered on both sides using gauze. The gauze may be soaked with sterile saline solution. To maintain and identify the orientation of the amnion, different color gauze may be used. For example, the fetal side may be covered with a plain white gauze, while the maternal side may be covered with a white gauze having a blue stripe. The amnion may be laid flat with the maternal side on the gauze having the blue stripe, and the epithelial (fetal) side up. The plain white gauze is placed over the epithelial layer. The upper left hand corner of the gauze-amnion-gauze stack may be cut off. The gauze may function as a wicking agent that draws moisture to the amnion during processing and storage, thereby preserving the integrity of the amnion.

In another embodiment, the amnion is placed on a gauze/synthetic mesh with the epithelial side down. The gauze/synthetic mesh is laid on a sticky backing material that holds the gauze/synthetic mesh in place. A clear or semi-clear covering is placed over the top of the maternal side of the amnion and adheres to the edges of the sticky backing material. The covering holds the amnion in place with minimal pressure. It will be understood that, if desired, the amnion can be similarly packaged with the maternal side down.

The amnion covering and packaging embodiments discussed above allow the physician to view the amnion patch and allow resizing the amnion patch in the package while maintaining the orientation of the amnion patch. In addition, the coverings provide support for the amnion patch for transfer to the surgical site. For example, if the amnion is covered with gauze on both sides, the physician may pull the gauze from the side to be applied to the surgical site and use the gauze on the opposite side to hold and manipulate the amnion patch during application. The gauze can be removed from the back of the amnion after the opposing side is applied to the surgical site. If a sticky backing material is used, the clear/semi-clear covering can be removed and the gauze/backing can be used to hold and manipulate the amnion for application to the surgical site. The gauze/backing can be removed after the opposing side has been applied to the surgical site.

It will be understood that the invention is not limited in this regard, and any appropriate material may be used to cover the treated amnion. For example, one or both sides of the amnion may be covered with cloth, plastic, coated or uncoated paper, an adhesive backing, mylar film or combinations thereof. In addition, any convenient means of marking the cover material to maintain and identify the orientation of the amnion may be used.

The covered amnion is cut into surgical patches of any desired size for a particular application. A rotary type cutting tool may be used to cut the gauze covered amnion. A grooved cutting board may be used to aid in cutting a straight and correctly sized patch. In another embodiment, the amnion is cut by free hand using a scalpel and ruler to achieve the desired size. In preferred embodiments, the amnion is cut to 2×4 cm, 4×8 cm, or 6×10 cm patches. The amnion should be cut at a size that is no less than the total area required for each patch.

The cut amnion patches between the gauze or other covering material are folded in half and packaged. The amnion patches are packaged in sterile saline solution to keep the tissue moist until use. In a preferred embodiment, the amnion patches are packaged in foil lined mylar pouches containing about 1 cc of sterile saline solution. The saline solution ensures that the amnion remains moist and facilitates implantation by the physician. In addition, the saline solution can eliminate or minimize the need to rehydrate the amnion prior to use. The foil lined mylar packages are heat sealed and the packaged amnion patches are terminally sterilized by irradiation, preferably by E-Beam sterilization in a range of 10-35 KGy. The packaged, sterilized amnion typically is expected to have a minimum shelf life of one year, and a maximum of five years, at ambient temperature.

The tissue is packaged such that the end user can unfold the tissue and identify the orientation of the tissue from the gauze or other material covering the amnion. The user can remove the gauze from the side of the amnion to be placed on the wound, place the amnion on the wound site, and remove the second gauze.

### Evaluation of the Anti-Adhesion Properties of Amnion

An evaluation of the properties of gluteraldehyde treated anti-adhesion amnion patches prepared in accordance with the process described above was performed using a sheep model. In the sheep laminectomy study, a total of fourteen adult sheep were used. The laminectomies were performed at the L2/L3 and L4/L5 sites. All animals were euthanized at 8 weeks post implant and the entire section of spine from L1 thru L6 was excised and sent for decalcification and histology. Following euthanization of the sheep, a pathology review of histology slides of the spines was conducted. The key for evaluation of the histology slides is provided in Table 1.

TABLE 1
Key for Evaluation on Histology Slides for Sheep Study

| Barrier Characteristics | 0 | 1 | 2 | 3 |
|---|---|---|---|---|
| Membrane | not present | present | NA | NA |
| membrane intact | intact/ | fragmented | NA | NA |
| | continuous | | | |
| inflammation- | no inflam- | some | more | exten- |
| lymphocytic | mation | | | sive |
| inflammation- | no inflam- | some | more | exten- |
| granulmatous | mation | | | sive |
| fibrosis | no fibrosis | some | more | exten- |
| | | | | sive |
| adhesion-fill fibrosis- | no adhesion | minor focal | extensive | NA |
| membrane | | adhesion | adhesion | |
| adhesion-Dural- | no adhesion | minor focal | extensive | NA |
| membrane | | adhesion | adhesion | |

### Example 1

### Amnion Patch Treated with Glutaraldehyde Implanted with the Maternal Side Towards the Dura

Gluteraldehyde treated amnion patches were prepared using the method described above, with the patches treated in 1% gluteraldehyde solution for 15 minutes. Three saline rinses as described above were performed before and after the gluteraldehyde treatment. In this example, the amnion treated patches were implanted with the maternal side of the amnion towards the dura.

The pathologist's review revealed the following. The laminectomy site was filled completely with reactive bone, reactive fibrocartilaginous tissue and reactive fibrosis. The reactive bone was organized and extended from the ends of the adjacent vertebral bodies. Superficially, there were ligaments and skeletal muscle bundles with regional muscle fiber atrophy and endomysial fibrosis. Separated by a distinct linear clear zone was an underlying membrane with a simple layer of flattened cuboidal epithelioid cells upon a hypocellular fine fibrillar membrane. The membrane curled superficially at the edges of the bone defect into the reactive bone and fibrous filling of the defect. Within the space, there was a thin fibrous membrane with hemorrhage immediately adjacent to the bone (periosteum). The membrane lay upon and was continuous with the dura mater. There was modest associated lymphocytic and granulomatous inflammation. The dura had no to minimal thickening fibrosis. There was an intact subdural space that contained a partial membrane-like structure that was composed of loose disorganized fibrillar to granular material with embedded RBCs, collagenous fragments and rare bone fragments. This membrane surrounded nerve roots. It overlay the leptomeninges (pia mater). There were no underlying spinal cord changes.

A summary of the pathologist's evaluation of the spines implanted with the gluteraldehyde treated amnion patches is provided in Table 2. For comparison, a summary of the finding of the pathologist for spines implanted with non-treated amnion patches is provided in Table 3.

TABLE 2
Glutaraldehyde treated amnion patch, Maternal side towards the Dura

| | | | | | | | | adhesion- | adhesion- |
|---|---|---|---|---|---|---|---|---|---|
| | | Section | | membrane | inflammation- | inflammation- | | fill fibrosis- | Dural- |
| sheep | lam site | Level | membrane | intact | lymph | granulomatous | fibrosis | membrane | membrane |
| 1 | A | cran | 1 | 1 | 1 | 1 | 1 | 0 | 2 |
| | | middle | 1 | 1 | 1 | 1 | I | 0 | 2 |
| | | caudal | 1 | 1 | 1 | 1 | 1 | 0 | 2 |
| | B | cran | 1 | 1 | 0 | 0 | 1 | 0 | 2 |
| | | middle | 1 | 1 | 0 | 0 | 1 | 0 | 2 |
| | | caudal | 1 | 1 | 0 | 0 | 1 | 0 | 2 |
| 3 | A | cran | 1 | 1 | 1 | 0 | 1 | 0 | 2 |
| | | middle | 1 | 1 | 1 | 0 | 1 | 0 | 2 |
| | | caudal | 1 | 1 | 1 | 0 | 1 | 0 | 2 |
| | B | cran | 1 | 1 | 1 | 0 | 1 | 0 | 2 |
| | | middle | 1 | 1 | 2 | 1 | 1 | 0 | 2 |
| | | caudal | 1 | 0 | 1 | 1 | 1 | 0 | 2 |
| 9 | A | cran | 1 | 1 | 1 | 0 | 0 | 0 | 1 |
| | | middle | 1 | 1 | 0 | 0 | 1 | 0 | 2 |
| | | caudal | 1 | 1 | 0 | 0 | 1 | 0 | 2 |
| 11 | A | cran | 1 | 0 | 3 | 2 | 3 | 2 | 2 |
| | | middle | 1 | 0 | 3 | 2 | 3 | 2 | 2 |
| | | caudal | 1 | 1 | 3 | 2 | 2 | 0 | 2 |

TABLE 3
Non-treated amnion patch, Maternal side towards the Dura

| | | | | | | | | adhesion- | adhesion- |
|---|---|---|---|---|---|---|---|---|---|
| | | Section | | membrane | inflammation- | inflammation- | | fill fibrosis- | Dural- |
| sheep | lam site | Level | membrane | intact | lymph | granulomatous | fibrosis | membrane | membrane |
| 2 | B | cran | 0 | na | 0 | 0 | 0 | 0 | 0 |
| | | middle | 1 | 0 | 2 | 2 | 2 | 0 | 2 |
| | | caudal | 1 | 0 | 2 | 2 | 2 | 0 | 2 |
| 4 | A | cran | 0 | na | 0 | 0 | 2 | 2 | 2 |
| | | middle | 0 | na | 0 | 0 | 1 | 2 | 2 |
| | | caudal | 0 | na | 0 | 0 | 2 | 2 | 2 |
| 8 | A | cran | 1 | 1 | 1 | 1 | 2 | 0 | 2 |
| | | middle | 1 | 1 | 2 | 3 | 2 | 1 | 2 |
| | | caudal | 1 | 1 | 2 | 3 | 2 | 1 | 2 |
| | B | cran | 1 | 1 | 1 | 3 | 2 | 2 | 2 |
| | | middle | 1 | 1 | 3 | 3 | 2 | 2 | 2 |
| | | caudal | 1 | 1 | 3 | 3 | 2 | 2 | 2 |
| 10 | B | cran | 1 | 1 | 3 | 1 | 2 | 1 | 2 |
| | | middle | 1 | 1 | 2 | 1 | 2 | 1 | 2 |
| | | caudal | 1 | 1 | 1 | 0 | 2 | 0 | 2 |
| 12 | B | cran | 1 | 0 | 3 | 3 | 3 | 2 | 2 |
| | | middle | 1 | 0 | 3 | 3 | 3 | 2 | 2 |
| | | caudal | 1 | 0 | 3 | 3 | 3 | 2 | 2 |

### Example 2

### Amnion Patch with Non-Treated Amnion Patches with the Fetal Side Towards the Dura

A group of sheep were implanted with amnion patches that were not treated with gluteraldehyde with the fetal side of the amnion toward the dura. The pathologist's review revealed the following. Embedded within the abundant reactive fibrosis filling the bone defect was a partial plicated membrane that was eosinophilic and acellular. It had “fragmentation” with fibrosis filling the defects. There was intimately associated mild lymphocytic inflammation forming a single lymphofollicular structure. There was extensive fibrosis of the dura that was continuous with the filling fibrosis. A summary of the findings is presented in Table 4.

TABLE 4
Non-treated amnion patch, Fetal side towards the Dura

| | | | | | | | | adhesion- | adhesion- |
|---|---|---|---|---|---|---|---|---|---|
| | | Section | | membrane | inflammation- | inflammation- | | fill fibrosis- | Dural- |
| sheep | lam site | Level | membrane | intact | lymph | granulomatous | fibrosis | membrane | membrane |
| 2 | A | cran | 1 | 1 | 2 | 2 | 3 | 2 | 1 |
| | | middle | 1 | 0 | 2 | 2 | 3 | 2 | 2 |
| | | caudal | 1 | 0 | 2 | 2 | 3 | 2 | 2 |
| 4 | B | cran | 1 | 1 | 3 | 2 | 3 | 2 | 2 |
| | | middle | 1 | 1 | 2 | 2 | 3 | 2 | 2 |
| | | caudal | 1 | 1 | 2 | 2 | 3 | 2 | 2 |
| 5 | A | cran | 1 | 0 | 2 | 0 | 3 | 2 | 2 |
| | | middle | 1 | 0 | 2 | 0 | 3 | 2 | 2 |
| | | caudal | 0 | na | 1 | 0 | 3 | 2 | 2 |
| | B | cran | 1 | 0 | 2 | 0 | 3 | 2 | 2 |
| | | middle | 1 | 0 | 2 | 1 | 3 | 2 | 2 |
| | | caudal | 1 | 0 | 1 | 0 | 3 | 2 | 2 |
| 10 | A | cran | 1 | 1 | 2 | 3 | 3 | 2 | 2 |
| | | middle | I | 0 | 2 | 2 | 3 | 2 | 2 |
| | | caudal | 1 | 0 | 3 | 2 | 2 | 2 | 2 |
| 12 | A | cran | 1 | 1 | 3 | 3 | 3 | 2 | 2 |
| | | middle | 1 | 1 | 3 | 3 | 3 | 2 | 2 |
| | | caudal | 1 | 1 | 3 | 3 | 2 | 2 | 2 |

### Example 3

### Glutaraldehyde Treated Amnion Patch with the Fetal Side Towards the Dura

Gluteraldehyde treated amnion patches were prepared using the method described above, with the patches treated in 1% gluteraldehyde solution for 15 minutes. Three saline rinses as described above were performed before and after the gluteraldehyde treatment. In this example, the amnion treated patches were implanted with the fetal side of the amnion towards the dura.

The pathologist's review of the histology slides revealed the following. The laminectomy site was filled completely with reactive bone, reactive fibrocartilaginous tissue and reactive fibrosis. The reactive bone was organized and extended from the ends of the adjacent vertebral bodies. Superficially there were ligaments and skeletal muscle bundles with regional muscle fiber atrophy and endomysial fibrosis. There was a distinct linear eosinophilic membrane lined by a simple layer of flattened cuboidal epithelioid that was on the dural side of the membrane. There was an incomplete narrow cleared space between the membrane and the dura. The space contained protein fluid and moderate numbers of macrophages and few multinucleated inflammatory cells aligning on the epithelial lining. The membrane curled superficially at the edges of the bone defect into the reactive bone and fibrous filling of the defect. The defect fibrosis/bone production abuts and was continuous with the outer portion of the membrane. The dura had mild thickening fibrosis. There was scant lymphocytic inflammation near the edges of the membrane within the reactive fibrosis. There was an intact subdural space that contained a partial membrane-like structure that was composed of loose disorganized fibrillar to granular material with embedded RBCs, collagenous fragments and rare bone fragments. This membrane surrounded nerve roots. It overlay the leptomeninges (pia mater). There were no underlying spinal cord changes.

A summary of the pathologist's evaluation of the spines implanted with the gluteraldehyde treated amnion patches is provided in Table 5.

TABLE 5
Glutaraldehyde treated amnion patch, Fetal side towards the Dura

| | | | | | | | | adhesion- | adhesion- |
|---|---|---|---|---|---|---|---|---|---|
| | | Section | | membrane | inflammation- | inflammation- | | fill fibrosis- | Dural- |
| sheep | lam site | Level | membrane | intact | lymph | granulomatous | fibrosis | membrane | membrane |
| 6 | A | cran | 1 | 1 | 1 | 2 | 1 | 2 | 1 |
| | | middle | 1 | 1 | 0 | 2 | 1 | 2 | 1 |
| | | caudal | 1 | 1 | 0 | 1 | 1 | 2 | 1 |
| | B | cran | 0 | na | 0 | 0 | 0 | 0 | 0 |
| | | middle | 1 | 1 | 0 | 1 | 0 | 2 | 0 |
| | | caudal | 1 | 1 | 0 | 1 | 2 | 2 | 0 |
| 9 | B | cran | 1 | 1 | 0 | 2 | 3 | 2 | 1 |
| | | middle | 1 | 1 | 0 | 2 | 3 | 2 | 0 |
| | | caudal | 1 | 1 | 1 | 2 | 3 | 2 | 0 |
| 11 | B | cran | 1 | 0 | 1 | 2 | 1 | 2 | 1 |
| | | middle | 1 | 1 | 3 | 3 | 2 | 2 | 1 |
| | | caudal | 1 | 1 | 3 | 3 | 2 | 2 | 0 |
| 13 | A | cran | 1 | 1 | 1 | 1 | 2 | 2 | 0 |
| | | middle | 1 | 1 | 1 | 2 | 2 | 2 | 0 |
| | | caudal | 1 | 1 | 1 | 2 | 2 | 2 | 1 |

### Example 4

### Control

The pathologist's review of the control revealed a bone defect filled with reactive tissue. There was a dura that was thickened and was continuous with the filling reactive changes from the defect. There was no membrane. The subdural space was intact. The spinal cord appeared normal.

(Sham/Control) Cranial with modest embedded loose lymphocytic and granulomatous inflammation within the fibrosis.

### Use of Amnion Anti-Adhesion Barrier

In use, the AAB is placed on the wound or the surgical site with the maternal side of the amnion in contact with the wound or surgical site. The AAB wound dressing may be used, for example, for surgery involving the spine, knee, shoulder or child birth, trauma related wounds or injuries, cardiovascular procedures, angiogenesis stimulation, brain/neurological procedures, burn and wound care, ophthalmic procedures or in any other procedure where an anti-adhesion barrier is desirable. In cases where fixation of the AAB is not required (spine, child birth, general surgery etc.), the AAB can be simply placed in the surgical site and held in place by the patient's musculature and skin throughout the recovery process. Alternatively, in some cases sutures or staples may be required to hold the AAB in place. In these cases, care must be taken so as not to tear or rip the AAB.

The AAB material may also be used to cover an implant to give the implant anti-adhesion properties that would be highly advantageous in terms of low surgical time, such as covering metal or biomaterial fixation or support devices. If desired, the amnion may be treated to provide for the delivery of a variety of antibiotics, anti-inflammatory agents, growth factors and/or other specialized proteins or small molecules. In addition, the amnion may be combined with a substrate (corion component, sterile gauze, sterile polymer material or other tissue or biomaterial) to increase the strength of the AAB dressing for sutures or increased longevity of an implant.

In other embodiments, the amnion tissue could be solubilized or ground up into a powder, gel or liquid and sprayed or poured on the device or tissue implant to prevent tissue adhesion.

One skilled in the art will recognize that numerous variations or changes may be made to the process described above without departing from the scope of the present invention. Accordingly, the foregoing description of preferred embodiments and following examples are intended to describe the invention in an exemplary, rather than a limiting sense.

We claim:

1. A method for producing an anti-adhesion wound dressing from human birth tissue comprising the steps of:
(a) obtaining human birth tissue comprising amnion, chorion, umbilical cord and placenta and removing the placenta and umbilical cord;
(b) separating the amnion from the chorion;
(c) rinsing the amnion with a sterile saline solution;
(d) immersing the amnion in a 1% glutaraldehyde solution for a period of up to 15 minutes;
(e) rinsing the glutaraldehyde treated amnion in a sterile saline solution;
(f) covering the amnion on top and bottom with a cover material;
(g) cutting the covered amnion to the desired size;
(h) packaging the cut amnion and terminally sterilizing the packaged amnion using irradiation.

2. The method of claim 1, wherein the step of rinsing the amnion in sterile saline solution prior to immersing the amnion in glutaraldehyde comprises three rinse cycles in 0.9% NaCl solution for a maximum period of 5 minutes per rinse cycle.

3. The method of claim 2, wherein the step of rinsing the amnion in sterile saline solution following immersing the amnion in glutaraldehyde comprises three rinse cycles in 0.9% NaCl solution for a maximum period of 5 minutes per rinse cycle.

4. The method of claim 3, wherein the cover material is selected from the group consisting of gauze, cloth, coated paper, uncoated paper, adhesive backing, plastic, mylar film, and combinations thereof.

5. The method of claim 4, wherein the cover material is gauze.

6. The method of claim 4, further comprising the step of identifying the orientation of the amnion prior to treating the amnion.

7. The method of claim 6, wherein the identification of the orientation of the amnion is by one of colored clips, stamping, ink marking or marked adhesive backing.

8. The method of claim 1, wherein the container is a foil lined mylar pouch that is heat sealed.

9. The method of claim 8, wherein the amnion is terminally sterilized using E-Beam gamma irradiation at between 10-35 kGy.

10. A method for producing an anti-adhesion wound dressing from human birth tissue comprising the steps of:
(a) obtaining human birth tissue comprising amnion, chorion, umbilical cord and placenta and removing the placenta and umbilical cord;
(b) separating the amnion from the chorion;
(c) identifying the fetal side and maternal side orientation of the amnion;
(d) rinsing the amnion in sterile 0.9% NaCl solution in three rinse cycles for a maximum period of 5 minutes per rinse cycle;
(e) immersing the amnion in a 1% glutaraldehyde solution for a period of up to 15 minutes;
(f) rinsing the amnion in sterile 0.9% NaCl solution in three rinse cycles for a maximum period of 5 minutes per rinse cycle;
(g) covering the amnion on top and bottom with a gauze cover material wherein the gauze cover material is marked to maintain and identify the fetal side and maternal side orientation of the amnion;
(h) cutting the covered amnion to the desired size;
(i) packaging the cut amnion in a foil lined mylar pouch containing sterile saline solution and heat sealing the pouch;
(j) terminally sterilizing the packaged amnion using E-beam gamma irradiation at between 10-35 kGy.

11. A method for dressing a wound using an amnion anti-adhesion wound barrier comprising the steps of:
(a) providing an amnion anti-adhesion wound barrier produced by the process of claim 10; and
(b) applying the amnion anti-adhesion wound barrier to a wound site.

12. The method of claim 11. wherein the wound site is selected from the group consisting of surgery involving the spine, knee, shoulder, or child birth, trauma related wounds or injuries, cardiovascular procedures, angiogenesis stimulation, brain/neurological procedures, burn and wound care, and ophthalmic procedures.

13. The method of claim 12, wherein the epithelial side of the amnion anti-adhesion wound barrier is placed in contact with the wound site.

14. The method of claim 12, wherein the stromal side of the amnion anti-adhesion wound barrier is placed in contact with the wound site.

15. The method of claim 12, wherein the amnion anti-adhesion wound barrier is secured to the wound site using bioglue, sutures or staples.

16. The method of claim 12, wherein the wound site is surgery of the spine and the stromal side of the amnion anti-adhesion wound barrier is applied to the dura.

17. An amnion anti-adhesion wound barrier produced by the process of claim 11.
